(19) United States
(12) Patent Application Publication
(10) Pub. No.: US 2020/0286610 A1
(43) Pub. Date: Sep. 10, 2020

(54) METHOD AND A DEVICE FOR USE IN A PATIENT MONITORING SYSTEM TO ASSIST A PATIENT IN COMPLETING A TASK

(71) Applicant: KONINKLIJKE PHILIPS N.V., EINDHOVEN (NL)

(72) Inventors: Frank WARTENA, Eindhoven (NL); Muhammad ASIM, Eindhoven (NL); Charles Leonardus Cornelius Maria KNIBBELER, Eindhoven (NL)

(21) Appl. No.: 16/883,381

(22) Filed: May 26, 2020

Related U.S. Application Data
(63) Continuation-in-part of application No. 14/422,950, filed on Feb. 20, 2015, now abandoned.
(60) Provisional application No. 61/695,048, filed on Aug. 30, 2012.

(51) Int. Cl. G16H 20/13; G06K 9/62; G08B 21/22; G06N 3/02

(52) U.S. Cl. CPC G16H 20/13; G06N 3/02; G08B 21/22; G06K 9/6215

(57) ABSTRACT

In some embodiments, a proximity of a patient to a medical device (e.g., a medication dispensing device in which the patient removes a dose of medication that has been dispensed therefrom) may be determined, and a medical status associated with the patient is determined. Based on the determined proximity and the medical status, an alert, including a triggering element, is generated. The alert includes a first alarm type and a second, different alarm type. Upon activation of the triggering element, the medical device is monitored to confirm initiation and occurrence of a physical action (e.g., dispensing and removal of medication) using the medical device. Based on the monitoring, the medical status associated with the patient is updated. A patient device and/or interface may be utilized for the proximity determination and/or generating the alerts.

### CROSS REFERENCE TO RELATED APPLICATIONS

This application is a continuation-in-part of U.S. patent application Ser. No. 14/422,950, filed Feb. 20, 2015, which is the U.S. National Phase application under 35 U.S.C. § 371 of International Application No. PCT/IB2013/058149, filed on Aug. 30, 2013, which claims the benefit of U.S. Provisional Patent Application No. 61/695,048, filed on Aug. 30, 2012. Each of these applications are hereby incorporated by reference herein in its entirety.

### BACKGROUND

### Field

The present disclosure is generally relates to a method and system in which a patient is provided with a device to assist them with monitoring and managing a health condition, and, in particular, relates to managing dispensing of medication and/or medical actions for the patient to complete.

### Description of Related Art

Various patient monitoring (personal telehealth) systems have been proposed. Conventionally such telehealth systems involve a patient being provided with one or more measurement devices and/or another patient device, such as a computer, smart phone or tablet computer, so the patient can take measurements of their physiological characteristics, complete surveys or questionnaires on their current status or compliance with a healthcare regimen, view educational or instructional content related to the patient's health condition, and/or remind the patient when they are to take the next dose of medication.

Current telehealth systems assign and present tasks to patients in order to assist the patient in complying with a prescribed medication and/or exercise regimen. Such tasks might include “measure your weight”, “take medication,” or “complete the survey on diet”. Through the presentation of these tasks, patients are made aware of what actions they need to take to manage their health condition.

### SUMMARY

Certain embodiments described herein relate to methods, apparatuses, and/or systems for facilitating dispensing of medication by a medication dispensing system.

The medication dispensing system includes one or more processors executing computer program instructions that, when executed, cause the one or more processors to: determine a proximity of a patient to a medication dispensing device associated with the patient, the medication dispensing device being configured to dispense a medication for the patient; determine a medication status associated with the patient, the medication status being related to the medication that is dispensed by the medication dispensing device; and generate, via the medication dispensing device, an alert including a dispensing triggering element based on the proximity of the patient and the medication status such that: (i) the alert includes a first alarm type in response to the proximity satisfying a proximity threshold and the medication status satisfying a first timing threshold; and (ii) the alert includes a second alarm type in response to the proximity satisfying the proximity threshold and the medication status satisfying a second timing threshold different from the first timing threshold. The one or more processors are further caused to: detect patient activation of the dispensing triggering element of the alert; instruct, based on the detection, the medication dispensing device to dispense a predetermined dose of medication from a storage area of the medication dispensing device; monitor, via one or more sensors of the medication dispensing device, the medication dispensing device for occurrence of the dispensing of the predetermined dose and occurrence of a removal of the predetermined dose from the medication dispensing device; and update, based on the monitoring indicating occurrence of the removal of the predetermined dose, the medication status associated with the patient.

In some embodiments, the medication dispensing device includes a receiving area and the predetermined dose of medication is dispensed from a storage area to the receiving area. In some embodiments, the one or more sensors are configured to determine removal of the predetermined dose from the receiving area of the medication dispensing device.

In some embodiments, a patient device is associated with the patient that establishes communications with the one or more processors, and the proximity of the patient device to the medication dispensing device is determined via the sensor therein.

In some embodiments, one alarm type is issued via the patient device, while the other alarm type is issued using the medication dispensing device.

One of the first alarm type and the second alarm type may be an audio alert, and the other may be a visual alert, in accordance with embodiments herein.

In some embodiments, physiological data (age, gender, and type and dose of medication being dispensed from the dispensing device) may be obtained by the one or processors. A plurality of users related to the patient based on the obtained physiological data may be determined, and the one or more processors are configured to: obtain, based on the plurality of users, combined response data indicating actual responses of the plurality of users to presentation of generated alerts including the dispensing triggering element; provide the combined response data related to the plurality of users to a neural network as reference feedback for the neural network's generation of alerts, the neural network configuring one or more layers of the neural network based the combined response data and the predicted generated alerts; subsequent to the configuration of the neural network, provide the physiological data related to the patient to the neural network to predict a response to the generated alert for the patient; and cause, via the medication dispensing device, presentation of the predicted response to the patient.

Some embodiments described herein relate to methods, apparatuses, and/or systems for facilitating a physical action for a patient to take or perform with respect to a medical device. Another aspect of this disclosure provides a method that includes: determining a proximity of a patient to a medical device, the medical device being arranged such that the patient performs a physical action therewith; determining a medical status associated with the patient, the medical status being related to the physical action for the patient to initiate with the medical device; and generating an alert including a triggering element based on the proximity of the patient and the medical status such that: (i) the alert includes a first alarm type in response to the proximity satisfying a proximity threshold and the medical status satisfying a first timing threshold; and (ii) the alert includes a second alarm type in response to the proximity satisfying the proximity threshold and the medical status satisfying a second timing threshold different from the first timing threshold. The method further includes: detecting patient activation of the triggering element of the alert; monitoring, via one or more sensors of the medical device, initiation and occurrence of the physical action by the patient using the medical device; and updating, based on the monitoring indicating occurrence of the physical action, the medical status associated with the patient.

In some embodiments, there are provided corresponding methods as well as a non-transitory computer-readable recording medium that stores therein a computer program product, which, when executed by a processor, causes the method disclosed herein to be performed.

Other aspects, features, and advantages of the present disclosure will become apparent from the following detailed description, the accompanying drawings, and the appended claims.

### BRIEF DESCRIPTION OF THE DRAWINGS

For a better understanding of the disclosure, and to show more clearly how it may be carried into effect, reference will now be made, by way of example only, to the accompanying drawings, in which:

FIG. 1A is a block diagram of a patient monitoring or telehealth system in which the disclosure may be implemented;

FIG. 1B is a block diagram of a medication dispensing system in which the disclosure may be implemented in accordance with one embodiment;

FIG. 2A is a flow chart illustrating a method of operating the medication dispensing system of FIG. 1B according to an embodiment of the disclosure;

FIG. 2B is a flow chart illustrating a method of operating a system including a medical device with sensor(s) according to another embodiment of the disclosure;

FIG. 2C is a flow chart illustrating a method of operating a patient device according to yet another embodiment of the disclosure;

FIG. 3 is an illustration of an exemplary data structure for a task according to an embodiment of the disclosure;

FIG. 4 is an illustration of exemplary task types in an embodiment of the disclosure;

FIGS. 5, 6 and 7 are illustrations of exemplary additional information that may be specified for some of the exemplary task types shown in FIG. 4;

FIG. 8 is a flow chart illustrating a method of operating a patient device according to a specific implementation of the disclosure;

FIG. 9 is a flow chart illustrating a method of operating a patient device according to another specific implementation of the disclosure;

FIG. 10 is a flow chart illustrating a method for facilitating a response to a generated alert for a patient in accordance with an embodiment;

FIG. 11 shows a system for facilitating a response to a generated alert, according to one or more embodiments.

FIG. 12 shows a machine learning model and data for configuring the machine learning model, according to one or more embodiments.

### DETAILED DESCRIPTION OF THE PREFERRED EMBODIMENT(S)

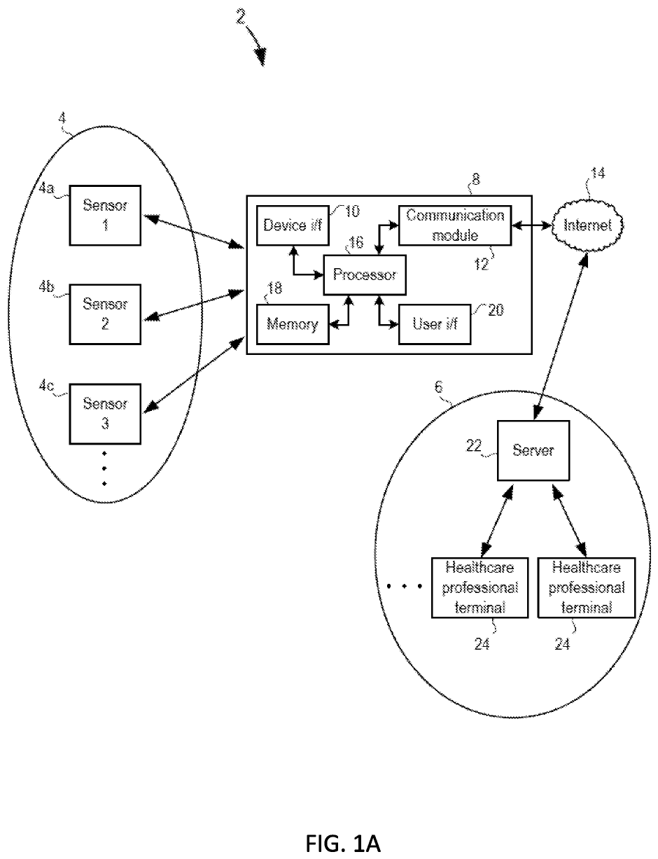

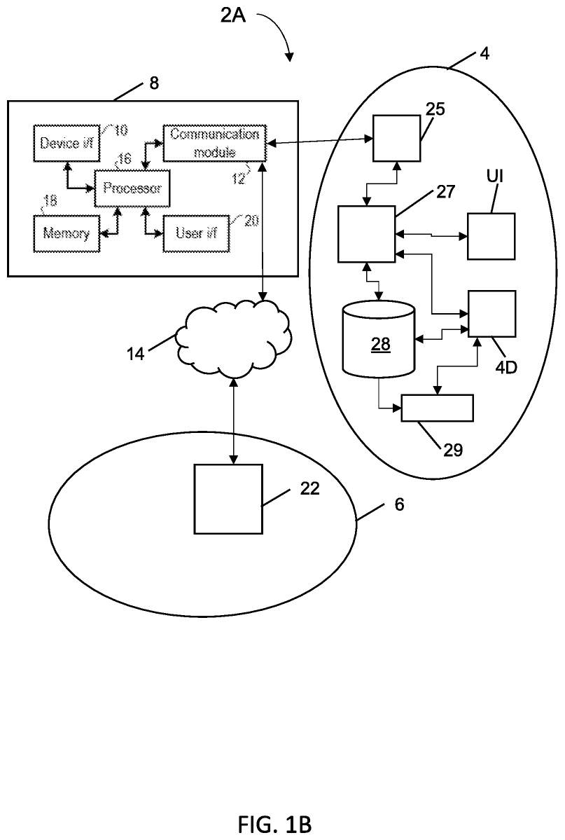

FIG. 1A illustrates an exemplary system 2 in which the disclosed features may be implemented. The system 2 includes one or more medical devices 4 that have sensor(s) associated therewith. One or more devices 4 may be provided and are used for monitoring and/or measuring physiological characteristics of the patient, in accordance with an embodiment. As an example, devices 4 may be medical measurement devices; e.g., in the exemplary illustrative embodiment of FIG. 1A, measurement device 4a may be a blood pressure monitor, measurement device 4b may be a glucometer and measurement device 4c may be a weighing scale, although it will be appreciated that other measurement devices may additionally or alternatively be used. In another embodiment, medical device 4 may be a medication dispensing device for dispensing a predetermined dose of medication (from a storage container containing a volume of medication for multiple doses of the medication) for a patient (or user) to take. The medication dispensing device (described in greater detail with reference to FIG. 1B later) may have one or more sensors associated therewith and/or provided on the device itself, e.g., for determining that a predetermined dose has been dispensed and for determining removal of the dispensed dose of medication by the patient. Medication status may be acquired and/or updated as part of a patient's health data. The health data acquired by one or more of these medical devices may then be transmitted to a remote healthcare service 6 for recording and/or analysis.

Whilst some medical devices 4 may be able to communicate directly with a healthcare service 6, typically a local apparatus 8, referred to herein as a telehealth hub, a patient device or a patient interface device, is used to collect and collate the data locally and then transmit the data to the healthcare service 6. The patient device 8 is communicatively coupled to the medical device 4. Thus each of the medical devices 4a-c are adapted to communicate with the telehealth hub 8. The medical devices 4a-c may be arranged to communicate with the telehealth hub 8 wirelessly via any suitable wireless protocol and/or via any suitable wired connection, i.e. the relevant medical device 4 could be plugged into the telehealth hub 8 in order to transfer data. The medical devices 4a-c may be arranged to transfer data automatically when new data is acquired, provided that a connection with the telehealth hub 8 is established, and/or to record measurement data in a local memory and then transmit it to the telehealth hub 8 periodically or at user initiated times, for instance when a connection with the telehealth hub 8 has been established.

In accordance with an embodiment, the patient device 8 and/or medical device 4 are configured to include alarm emitting devices, including, but not limited to, speaker(s), a vibration motor, and lights such as LEDs. Such alarm emitting devices may be associated with generating alert(s), which will become further evident based on the description below.

The telehealth hub 8 and medical devices 4a-c may all be located in an environment, such as a home environment of the patient, which is remote from a healthcare provider 6. The medical devices 4a-c may be used within the home environment although at least some medical devices may be portable and usable in other locations. Such data may be stored in the medical device 4 and then uploaded to the telehealth hub 8 when the patient returns home.

The telehealth hub 8 is configured to receive the data from the medical devices 4a-c through device interface(s) 10 and to transmit data using communication module 12 to the healthcare services 6, for instance via a network 14 such as the Internet or other wide area network (WAN). In an embodiment, the interface 10 may be provided as part of the medical device 4, e.g., a screen with an input device (e.g., keyboard, touch screen) provided directly on the device. For example, a medication dispensing device that is a self-contained system may include a touch screen as an interface thereon. In another embodiment, the interface 10 may be provided via another device that is in communication with the medical device 4, e.g., a smart phone, a smart watch, or an application (also known as an “app”) that is downloaded to a portable device using the network 14.

As noted above, it should be understood that the network 14 may include the Internet and/or other networks, such as local area networks, cellular networks, Intranets, near field communication, frequency (RF) link, Bluetooth™, Wi-Fi™, GPS, and/or any type(s) of wired or wireless network(s). Such examples are not intended to be limiting, and the scope of this disclosure includes embodiments in which the patient device 8, sensor(s) 4, processor(s), electronic storage, server 22, and/or client computing platform(s) are operatively linked via some other communication media.

The communication module 12 of the telehealth hub 8 may therefore provide a wired Internet or network connection although in other embodiments the communication module 12 in the telehealth hub 8 may be arranged to transfer data via a suitable mobile telephone network or any other type of communication network. In some embodiments, the telehealth hub 8 may be a dedicated telehealth hub apparatus but in other embodiments another device which is already suitable for remote communication may be configured to act as a telehealth hub, for instance a desktop or laptop computer, a mobile telephone, a tablet computer, a set-top entertainment box or the like. It will be appreciated that if the telehealth hub 8 is implemented in a portable device with a communications facility, such as a mobile telephone, smart watch, or portable computer, then the telehealth hub 8 may itself be used in other environments than the home environment.

In addition to the device interface(s) 10 and communication module 12, the telehealth hub 8 comprises a processor or control unit 16 that controls the operation of the telehealth hub 8, a memory module 18 (such as a hard disc, an optical storage medium or a non-volatile memory chip) that stores information and program code for use by the processor 16 in implementing the disclosure, and a user interface 20 which allows the patient to interact with the telehealth hub 8. The user interface 20 may comprise a display or screen (which may be a touch screen), one or more buttons or keys, a pointer-control device, such as a mouse or touchpad, and/or one or more audio speakers.

The healthcare service 6 comprises a server 22 that is connected to the (Internet or WAN) network 14 and that receives and stores patient data sent from the telehealth hub 8. The server 22 is connected to one or more computer terminals or devices 24 that are used by healthcare professionals to access the patient data.

It will be appreciated that although the medical devices 4 and the telehealth hub 8 are shown as separate devices in FIG. 1A and FIG. 1B, one or more of the medical devices 4 may be integrated into the telehealth hub 8, or vice versa.

In the illustrated telehealth system 2, the server 22 is the device that is responsible for implementing and executing the care plan of the patient. This care plan may include many tasks that the patient needs to complete in order to manage their health condition. These tasks may include measuring vital signs, other physiological characteristics, completing surveys, reviewing educational material, taking medication, etc. To implement the care plan, the server 22 sends information on the tasks the patient is to complete to the telehealth hub 8 via the (Internet or WAN) network 14.

The telehealth hub 8 (which may also be known as an application hosting device (AHD)) is the device with which the patient mainly interacts and this is the device that in most cases is responsible for conveying the task information received from the server 22 to the patient. In an embodiment, the server 22 is configured to obtain and/or generate information related to the patient and communicate the information to the medical device 4 and/or patient device 8.

FIG. 1B illustrates one exemplary embodiment for facilitating dispensing of medication by a medication dispensing system 2A. For purposes of clarity and brevity, like elements and components throughout the Figures are labeled with same designations and numbering as discussed with reference to FIG. 1A. Thus, although not discussed entirely in detail herein, one of ordinary skill in the art should understand that various features associated with system 2A are similar to those features previously discussed with respect to system 2 and FIG. 1A (above). Additionally, it should be understood that the features shown in each of the individual figures is not meant to be limited solely to the illustrated embodiments. That is, the features described throughout this disclosure may be interchanged and/or used with other embodiments than those they are shown and/or described with reference to. For example, while not explicitly depicted in FIG. 1B, it should be understood that the medication dispensing device 4 may include a memory and/or storage therein.

In some embodiments, the medication dispensing device 4 includes a user interface UI which allows the patient to interact with the device 4 and a communication module 25 to transmit data to communication module 12 and to the healthcare services 6, for instance via network 14. In an embodiment, the interface 10 may be provided as part of the medication dispensing device 4, e.g., a touch screen, or via another device as noted previously.

Medication dispensing device 4 includes a storage container 28 or storage area that contains a volume of medication (e.g., pills) therein. The volume of medication includes multiple doses of a medication for a user to take, e.g., a month's supply of medication. From the volume in the storage container 28, the medication dispensing device 4 is configured to select or withdraw a predetermined dose of the medication and direct the dose out of the storage container 28. Additionally, one or more sensor(s) 4D are provided as part of the medication dispensing device that is/are configured to monitor occurrence of the dispensing of a predetermined dose of medication from the storage container 28. In accordance with an embodiment, the medication dispensing device 4 may include an optional receiving area 29, such that the dispensing of the predetermined dose is directed from the storage container 28 to a receiving area 29 of the medication dispensing device. The receiving area 29 may be a receptacle or tray, for example, that receives the predetermined dose therein. Additionally, the one or more sensor(s) 4D may be configured to monitor occurrence of a removal of the predetermined dose (by the patient) from the receiving area 29.

The medication dispensing system 2A includes one or more processors for executing computer program instructions. The processor(s) may be processor 16 of the patient device 8 and/or a processor 27 associated with the medication dispensing device 4 in FIG. 1B. Processor 27 or control unit may be included for controlling the operation of the medication dispensing device. In another embodiment, the processor 16 and/or servers 22 may be used to control the medication dispensing device.

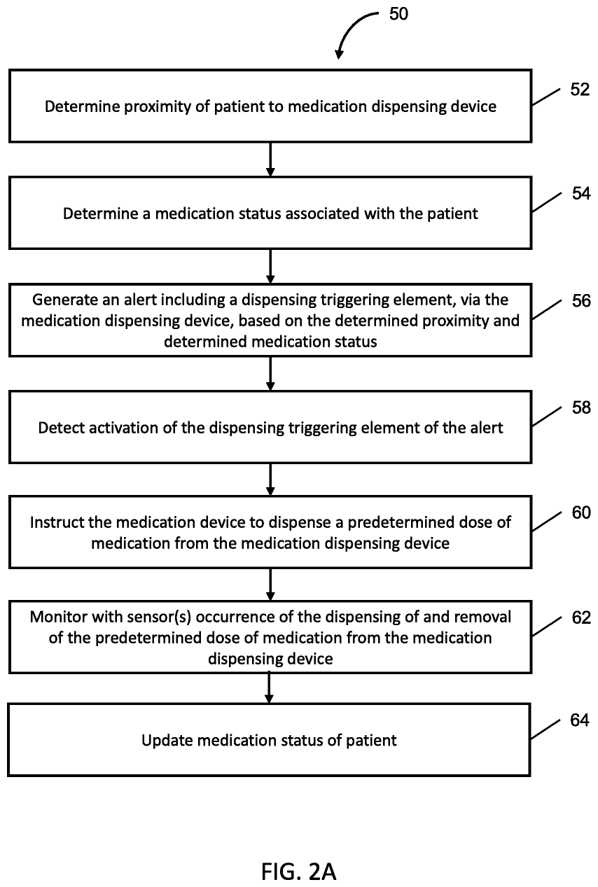

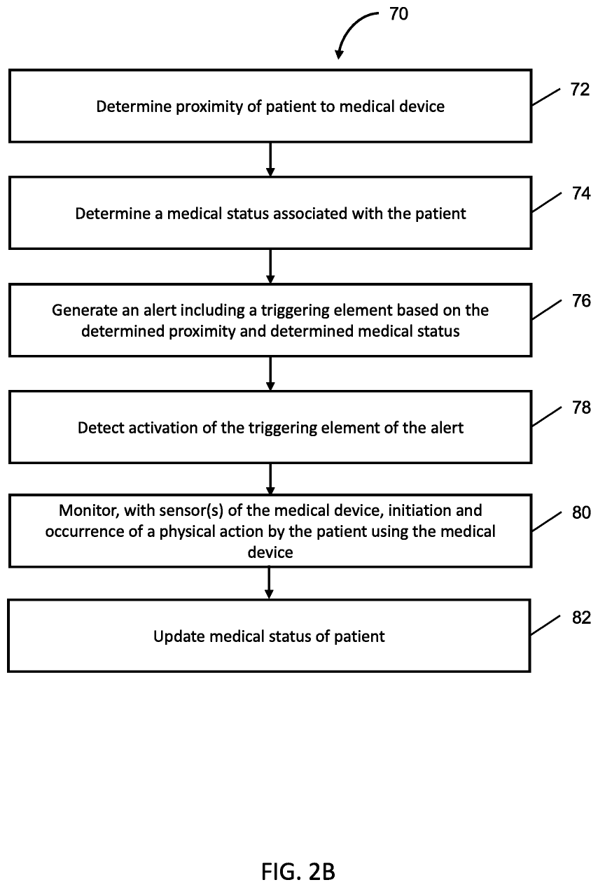

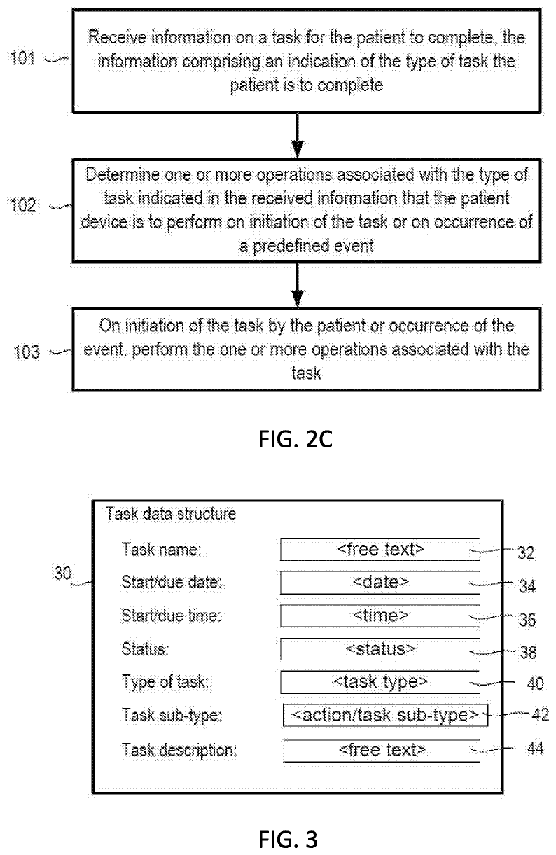

The computer program instructions, when executed, cause the one or more processors to implement a method 50 for operating the medication dispensing system 2A shown in FIG. 2A. In the method 50, the processors may be caused to: determine a proximity of a patient to the medication dispensing device 4 associated with the patient, as shown at 52. In some embodiments, patient device 8 is associated with the patient and establishes communications with the one or more processors, such that the proximity of the patient device 8 to the medication dispensing device 4 is determined via the sensor 4D therein. For example, in an embodiment, a patient's proximity and location may be determined via a Bluetooth and/or GPS connection between the patient device 8 and device 4.

The one or more processors may also determine a medication status associated with the patient, as shown at 54. The medication status is related to the medication that is dispensed by the medication dispensing device 4. For example, the medication status may include a determination that medication should be taken by a particular date and time and that said date and time and has not passed, whether or not the medication has been dispensed, the last date and time the patient removed and/or took the predetermined dose of medication, and/or if the patient has taken a dose of medication. In an embodiment, the determination for if a patient has taken a dose of medication is based on the sensor 4D determining the occurrence of dispensing of the predetermined dose. In one embodiment, the determination for if a patient has taken a dose of medication is based on the sensor 4D determining the occurrence of a removal of the predetermined dose from the receiving area of the medication dispensing device. In an embodiment, the determination for if a patient has taken a dose of medication is based on the sensor 4D determining both the occurrence of dispensing of the predetermined dose and a removal of the predetermined dose from the receiving area of the medication dispensing device. Such examples are not intended to be limiting, however.

Further, the method 50 includes, at 56, the one or more processors generating, via the medication dispensing device, an alert including a dispensing triggering element based on the proximity of the patient and the medication status. In an embodiment, the dispensing triggering element may include generating input button for a patient to press or click to confirm receipt of the alert and/or proceeding with dispensing of the dose. For example, the input button may be generated on a touch screen of a user interface on the medication dispensing device and/or on a screen of a patient device (e.g., a mobile phone, a smart watch, a computer, etc.), in accordance with embodiments herein. In an embodiment, the input button may include associated text, such as “dispense medication.” The generated alert may further include a first alarm type in response to the proximity satisfying a proximity threshold and the medication status satisfying a first timing threshold, and a second alarm type in response to the proximity satisfying the proximity threshold and the medication status satisfying a second timing threshold different from the first timing threshold. One of the first alarm type and the second alarm type may be an audio alert, and the other may be a visual alert, in accordance with embodiments herein. For example, the first alarm may be a reminder or indicator that is visually generated, e.g., providing an alert on a user interface or screen, vibrating or turning on a light or other indicator using the interface, the patient device, and/or medication dispensing device. The second alarm may be a further reminder that is auditory, e.g., emitting a sound such as a buzz, beep, and/or a pattern or series of audio signals using the interface, the patient device, and/or medication dispensing device. In an embodiment, the second alarm may include a visual indicator in addition to the audio indicator. In a particular embodiment, the first alarm and/or second alarm may include a progressive type of alarm, e.g., progressively lighting an LED from low brightness to highest brightness or from a first color to a second color, progressively issuing a series of vibrations, or progressively emitting a sound from a lower volume to higher volume (smooth to aggressive).

In some embodiments, one alarm type (e.g., first alarm) is issued via the patient device, while the other alarm type (e.g., second alarm) is issued using the medication dispensing device. In other embodiments, one alarm type is issued via the interface, while the other alarm type is issued using the medication dispensing device. In yet another embodiment, both alarm types are issued using both the patient device and the medication dispensing device.

The proximity threshold may be a predetermined distance and/or location that is preset by the system and/or set by the user. In an embodiment, the proximity threshold may be a distance (e.g., 10 feet) between the patient and the medication dispensing device. The distance may be determined between a patient device and the medication dispensing device via Bluetooth or GPS technology, for example, in accordance with an embodiment. In another embodiment, the location may be an area or room within a patient's house. The location and/or proximity of a patient device relative to the medication dispensing device may also determined, e.g., via Bluetooth or GPS technology. For example, if the medication dispensing device is positioned in a patient's bathroom or kitchen, and the patient walks by or enters this room, the proximity threshold may be met. The timing thresholds may be preset time periods for responding to the generated alert. For example, in an embodiment, a first timing threshold may be a preset time period of X number of minutes, and the second timing threshold may be another time period of Y number of minutes. In one embodiment, the first timing threshold and/or second timing threshold may be based on a specified date and time frame for a patient to take the dose of medication (or complete a task). In a particular embodiment, the first timing threshold may be a predetermined time period before the date/time specified for taking the medication, e.g., one hour before the patient should take the dose of medication. In an embodiment, the second timing threshold may be a time period that is less than the first, e.g., fifteen minutes. In one embodiment, the second timing threshold may be a predetermined time after the first timing threshold but also before the date/time specified for taking the medication, e.g., fifteen minutes before the patient should take the dose of medication. In yet another embodiment, the second timing threshold may be a time period after the first timing threshold.

As an example, once the patient is within the proximity threshold and has not yet taken a dose of medication, the system may generate the alert which includes a triggering element and generating the first alarm, e.g., a visual notification such as “please do not forget to take medication X before noon”, and then generate a second alarm fifteen minutes before noon that includes a series of emitting sounds to further remind the user to take the dose of medication.

In some embodiments, the second alarm type may be issued when the system determines (e.g., via sensor) that the medication has not yet been taken (i.e., removed from the medication dispensing device (and/or optionally from the receiving area)).

Turning back to FIG. 2A, in the method 50, the one or more processors are further caused to, as shown at 58, detect patient activation of the dispensing triggering element of the alert, and then, at 60, instruct, based on the detection, the medication dispensing device to dispense a predetermined dose of medication from the storage area 28 of the medication dispensing device. In one embodiment, at 60, based on the detection, the medication dispensing device is configured to dispense the predetermined dose from the storage container 28 to the receiving area 29. At 62, the processor(s) monitor, via one or more sensors 4D of the medication dispensing device, the medication dispensing device for occurrence of the dispensing of the predetermined dose and occurrence of a removal of the predetermined dose. In an embodiment, the removal of the predetermined dose may be from the medication dispensing device 4 and/or from the receiving area of the medication dispensing device 4. Based on the monitoring indicating occurrence of the removal of the predetermined dose (e.g., from the device 4 and/or from the receiving area 29), the medication status associated with the patient is updated using the processor(s).

In an embodiment, the update of the medication status includes recording a completion date and time that the disposed dose of medication was taken by the patient.

Some embodiments described herein relate to methods, apparatuses, and/or systems for facilitating a physical action for a patient to take or perform with respect to a medical device, which may be the aforementioned medication dispensing device or other medical devices as described above with respect to FIGS. 1A and 1B. FIG. 2B illustrates a method 70 of operating a system including a medical device with sensor(s), according to another embodiment of the disclosure, the method 70 including: determining a proximity of a patient to a medical device, as shown at 72, the medical device being arranged such that the patient performs a physical action therewith, and determining a medical status associated with the patient, at 74. The medical status may be related to the physical action for the patient to initiate with the medical device, e.g., step on a scale and activate the weight sensor, apply and activate the blood pressure cuff or monitor, etc. Further, at 76, an alert is generated that includes a triggering element based on the proximity of the patient and the medical status such that: (i) the alert includes a first alarm type in response to the proximity satisfying a proximity threshold and the medical status satisfying a first timing threshold; and (ii) the alert includes a second alarm type in response to the proximity satisfying the proximity threshold and the medical status satisfying a second timing threshold different from the first timing threshold. Examples of such alarm types and thresholds are described above with respect to FIG. 2A and the medication dispensing device and thus are not repeated here. The method further includes, as shown at 78, detecting patient activation of the triggering element of the alert; monitoring, via one or more sensors of the medical device, initiation and occurrence of the physical action by the patient using the medical device at 80; and updating, based on the monitoring indicating occurrence of the physical action, the medical status associated with the patient at 82.

In other embodiments, the features of the medication dispensing device and/or medical device are configured to perform additional determinations, detections, and/or actions, caused by the one or more processors of the system. In some embodiments, a completion date and time that a dispensed dose of medication has been removed by the patient (e.g., from the medication dispensing device, and/or optional receiving area 29) may be sensed by sensor 4D and/or the processor(s), and the completion date and time may be updated and/or recorded as part of the patient's information/data. The one or more processors and/or the medication dispensing device may be further configured to communicate the recorded completion date and time received from the sensor to the server 22.

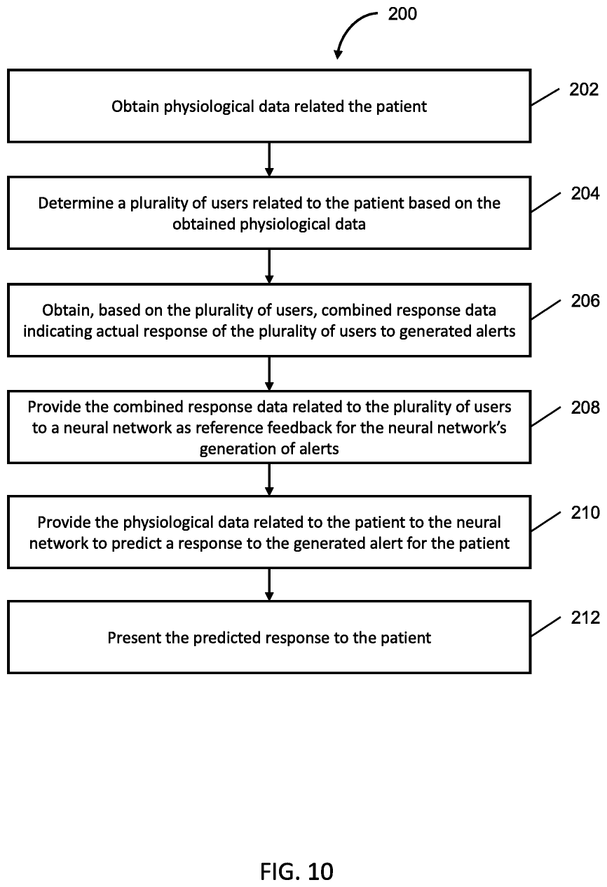

In some embodiments, the processor(s) and/or server may be configured to generate information related to the patient taking the predetermined dose of medication dispensed by the medication dispensing device by a specified date and time frame. As described in later embodiments, in some cases, a notification or alert may be generated and issued to the patient via the interface at a predetermined time before the specified date and time frame for taking the predetermined dose of medication, and, optionally, a follow up notification/alert may be issued via the interface and/or another device. In some embodiments, the specified date and time frame for the medication dispensing device to dispense a new predetermined dose of medication may be modified, e.g., via a neural network (as described with reference to FIGS. 10-12).

Other embodiments of this disclosure relate to a patient being reminded to complete one or more tasks. As described previously, current tools that have a task or reminder function for a user are able to help a patient with the timing of a task, but are not able to actively support the patient with the completion of the task itself. The tools are also not able to verify that the patient-selected status of a task (e.g. completed) is correct, or vice versa. Therefore, there is a need for an improved way of managing tasks within a patient monitoring or telehealth system 2 in accordance with some embodiments herein.

In accordance with the disclosure, the telehealth system 2 (or a part or parts of the telehealth system 2 such as the telehealth hub 8 and/or server 22) is provided with semantic understanding of tasks, for example including the actual action or actions that is or are required to be performed by the patient in order for the task to be completed. This semantic understanding is provided by specifying one or more additional parameters in the definition of a task in addition to the standard semantically-understood parameters which may include the owner (i.e. which patient the task is for), the start date, the due date (i.e. when the task must be completed by), priority (i.e. how important the task is), status (i.e. not yet completed, completed, etc.), and the non-semantically understood parameters which may include the title, summary and/or description of the task.

In preferred embodiments, the one or more additional parameters may be used to indicate the type of task to be completed by the patient. Examples of the types of tasks to be completed by the patient include taking a measurement of a physiological characteristic, completing a survey, reviewing educational content, taking the next dose of medication, etc. A set of task types may be predefined in the telehealth system 2, and a task type selected for each task that is to be completed by the patient. The task type may be selected at the time that the task or action is created by the patient, by the healthcare professional or by the server 22.

In further preferred embodiments, the one or more additional parameters also comprise a parameter that defines the sub-type of task to be completed by the patient. The available task sub-types may depend on the type of task selected (i.e. there may be a respective set of task sub-types applicable to one or more of the task types). As with the task types, a set of task sub-types may be predefined in the telehealth system 2, and a task sub-type selected once a task type has been selected for a particular task. The task sub-type may be selected at the time that the task is created by the patient, healthcare professional or the server 22. For example, for the ‘take a measurement’ task type, the task sub-type parameter may include the options: weight, blood pressure, pulse, glucose, etc.; for the ‘complete a survey’ task type, the task sub-type parameter may include the options: diet, general health, mental health, etc., or alternatively an address (such as a URL) or a storage location (for example a directory in the memory module 18 in the telehealth hub 8) where the survey is located; for the ‘review educational content’ task type, the task sub-type may include the options: exercise content, message from a healthcare professional, etc., or alternatively an address or location where the content is located; for the ‘take a medication dose’ task type, the task sub-type may include options for the type/name of the medication to be taken and/or the amount of medication to be taken and/or the date and time for the patient to take a dispensed amount of medication.

In alternative embodiments, it will be appreciated that rather than specifying a general task type and then specifying a task sub-type, a single larger set of task types may be defined that includes all of the information for the task required by the telehealth hub 8 to semantically understand the task. For example, the task types may include take a weight measurement, take a blood pressure measurement, complete a diet survey, complete a general health questionnaire (with, for example, a specified address or location for that questionnaire), review an exercise training video, take a 500 mg tablet of medication X, etc.

To provide the semantic understanding, a set of one or more operations is defined for the telehealth hub 8 to perform in connection with each type of task (and/or sub-type, where specified). The specified operation(s) will depend on the type (and sub-type) of task selected, and may be performed by the telehealth hub 8 as appropriate when the task is started or opened, or on occurrence of a predefined event (such as a measurement being received from a specific sensor 4 or a patient indicating they are ready to start or complete the task). Information on the operation or operations to be performed by the telehealth hub 8 in connection with each type of task (and/or sub-type) may be provided by the server 22 to the telehealth hub 8 along with the other information on the task (e.g. the due date, task name, type of task, etc.) or alternatively the telehealth hub 8 may store information on operations associated with each task (and/or sub-type) and determine the appropriate actions using the information on the task received from the server 22.

For example the telehealth system 2 may be assigned an operation to automatically start a workflow that helps guide the patient through the task that was assigned to them, e.g. when the patient opens or starts a task such as “measure your weight” on the patient device 8, the assigned operation may cause the device 8 to automatically navigate the input cursor on the display of the user interface 20 to the weight measurement entry field, or cause the device 8 to issue an instruction to the user to step on a medical device 4 such as a scale with a weight sensor, or when the user opens or starts the task “complete the survey on diet” the operation for the telehealth hub 8 could cause the hub 8 to automatically open the diet survey from the specified URL or memory location so the hub 8 is ready for the user to answer the questions.

In addition or alternatively, the specified operation(s) may cause the telehealth hub 8 to automatically check if a task has been completed, for example when the user has a task “measure your weight” the telehealth hub 8 may automatically mark that task as ‘completed’ if the telehealth hub 8 receives a new weight measurement from the weight sensor of the medical device 4 (the receipt of the weight measurement from the weight sensor of the medical device 4 being a predefined event as described above). Likewise, where the patient's medication is dispensed by an electronic device (e.g., a medication dispensing device like that in FIG. 1B) that is connected to the telehealth system 2, the telehealth hub 8 may mark a ‘take medication’ task as completed when it receives a signal from the sensor associated with medication dispensing device indicating that a dose of medication has been removed by the patient from the dispensing device.

In addition or alternatively to the above, the specified operation(s) may include sending or issuing reminders to the patient to complete the task. In this case, the reminders may be generated based on the task type (and sub-type specified), e.g. “please do not forget to take medication X before noon”.

The patient will benefit from this improved task management since it results in a smoother workflow for the patient as the hub-side of the task may be automatically started (e.g. survey retrieved and opened, educational content loaded), the taking of measurements detected automatically from the sensor(s) of the medical devices 4 in the system 2, and there being less unnecessary reminders to the patient (since a task may be marked completed automatically when the patient has completed their required action without the patient having to manually mark the task as completed).

The standardized format for task information is defined in RFC2445 (http://www.ietf.org/rfc/rfc2445.txt) section 4.6.2 as the to-do item:

“Description: A “VTODO” calendar component is a grouping of component properties and possibly “VALARM” calendar components that represent an action-item or assignment. For example, it may be used to represent an item of work assigned to an individual; such as “turn in travel expense today”.

This standard uses free text fields for parameters such as title, summary, description as described above. In some implementations of the disclosure, the one or more additional parameters or fields such as task type and sub-type may be added to the VTODO task definition, but it will be appreciated that similar approaches could be used for other task definition formats. The task type and sub-type parameters or fields are assigned a text-based, numerical or coded value from a predefined range of values (e.g. 0-9) each of which is associated with a respective type of task or task sub-type. The semantic understanding of the task by the telehealth hub 8 (and/or server 22, as appropriate) will be provided by associating one or more operations for the telehealth hub 8 to each of the values in the predefined range of values for the task type and sub-type fields.

In some embodiments, one of the values in the predefined range for the task type (or alternatively any value input in the field that does not take one of the values in the predefined range—in other words the task type field is used as a ‘free text’ field where only a small subset of all possible input values have a defined meaning) may be used to indicate an ‘unspecified’ task to the telehealth hub 8. This ‘unspecified’ task may be used for any task for the patient that is not one of the tasks predefined in the system 2. In this case, the free text entries in the “task type”, “task name” and/or “task description” fields may be used to indicate the type of task to be performed by the patient. It will be appreciated that the telehealth hub 8 will not have semantic understanding of this unspecified task and therefore will not be able to determine task-specific operations to perform to assist the patient in completing the task.

The flow chart in FIG. 2C illustrates a method of operating a patient device 8 according to an embodiment of the disclosure. In step 101, the patient device 8 receives information on one or more tasks for the patient to complete. The information comprises at least an indication of the type of task the patient is to complete, with the indicated task type being one found in a predefined set of task types. As noted above, the indication of the type of task the patient is to complete may comprise a numerical, text-based or coded value in a ‘task type’ field in a data structure used to define a task, with each type of task in the predefined set being associated with a respective numerical, text-based or coded value.

Step 101 may comprise receiving the task information from another device in the telehealth network 2, such as server 22 or healthcare professional terminal 24, or alternatively retrieving the task information from the memory module 18. The task information may be provided in the modified VTODO format discussed above, or in an alternative format.

Then, in step 102, the telehealth hub 8 determines one or more operations it is to perform to assist the patient in completing the task. Indications of the operations associated with the type of task may be received from the server 22 or healthcare professional terminal 24 with the task information itself, or alternatively the telehealth hub 8 may be programmed with the operations to be taken for each of the possible task types (and sub-types), in which case the telehealth hub 8 looks up the appropriate operations using the task type specified in the received task information.

The patient device 8 may also receive indications of when these one or more operations are to be performed (e.g. on initiation of the task, and/or on occurrence of a predefined event during the task), or again these indications could be programmed into the telehealth hub 8.

Then, in step 103, on initiation of the task by the patient, or on occurrence of the predefined event, the patient device 8 performs the one or more operations associated with the task.

FIG. 3 shows an exemplary data structure 30 for a task according to the disclosure. Information on the task may be communicated between the server 22 and telehealth hub 8 using this task structure 30. Data entry fields corresponding to this data structure 30 could be presented to a healthcare professional when the patient's healthcare regimen is being prepared and/or to a patient when setting their own tasks as part of the treatment or management regimen. It will also be appreciated that tasks may be set automatically by the telehealth system 2, in which case the server 22 or telehealth hub 8 may automatically populate the required fields.

Thus, in the data structure 30 shown in FIG. 3, there is a field 32 for a task name to be entered (e.g. measure weight, take medication, etc.). As in conventional task definitions, this field is free-text, meaning that the user (e.g. healthcare professional or patient) is free to input any text and/or other characters into this field (subject to a maximum permitted number of characters).

As with conventional tasks, the data structure 30 also comprises a start/due date field 34 that allows the user to select the date on which the task is to be started and/or completed by, a start/due time field 36 to allow the user to input the time by which the task is to be started and/or completed by, a status field 38 that indicates the status of the task (e.g. initially ‘not yet started’, although subsequently it could be ‘in progress’ or ‘completed’).

As indicated above, the start/due date 34, start/due time 36 and status 38 fields are fields that are semantically understood by the patient device 8 as they have a predefined set of available options (i.e. they are not free-text fields).

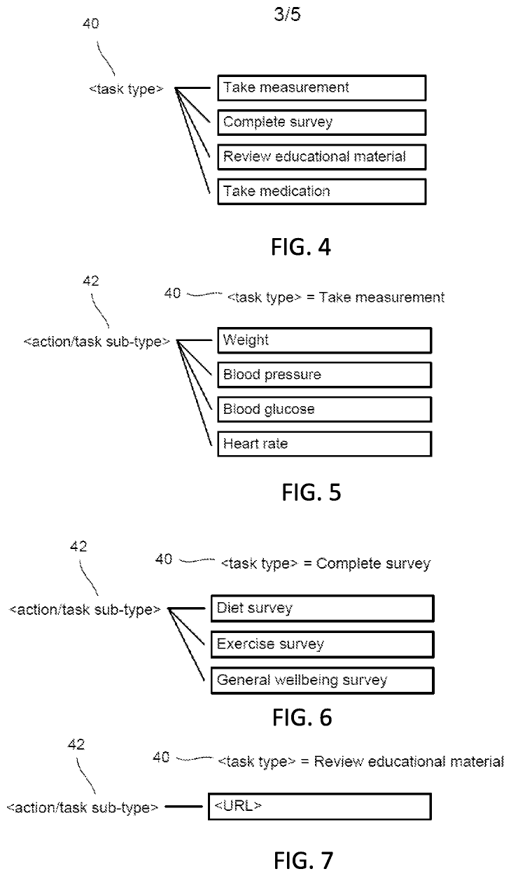

In accordance with an aspect of the disclosure, a further field 40 is provided which is used to indicate the type of task to be performed by the patient. As described above, there is a predefined (i.e. closed) set of task types which is known to the telehealth hub 8. FIG. 4 illustrates four exemplary values for the task type field 40 in accordance with the disclosure. The four task types comprise “Take measurement”, “Complete survey”, “Review educational material” and “Take medication”. It will be appreciated that each task type shown in FIG. 4 may be represented by a number or code that is input into the task type field 40, rather than inputting text such as “Take measurement”.

In some embodiments (for example in FIG. 4 where the task type field 40 only specifies the general nature of the task), the data structure 30 may comprise a further field 42 for allowing the task sub-type or action (e.g. weight, blood pressure, survey type or location, etc.) to be specified. As described in more detail below, the available parameter values for the task sub-type field 42 may depend on the task type entered into field 40.

FIG. 5 shows the possible task sub-type values when the task type specified in field 40 is “Take measurement”. Thus, the possible sub-types available for entry in field 42 are “weight”, “blood pressure”, blood glucose”, “heart rate”, etc.

FIG. 6 shows the possible task sub-type values when the task type specified in field 40 is “Complete survey”. Thus the possible sub-types available for entry in field 42 are “Diet survey”, “Exercise survey”, “General wellbeing survey”, etc. Alternatively (and similarly to that shown in FIG. 7 below), rather than presenting a preset list of available sub-types, field 42 may be a field that allows an address (e.g. URL) or location (e.g. in the memory module 18) to be specified where the survey is stored. Of course, it will be appreciated that, although not shown in FIG. 6, each of the task sub-types shown may separately have an associated address or location where each survey is located (which may be determined in step 102 above as part of determining the operation to be performed by the telehealth hub 8 in respect of the task type).

FIG. 7 shows the possible task sub-types when the task type specified in field 40 is “Review educational material”. In this case, the task sub-type field 42 allows the user to input an address or location where the educational material is stored. Alternatively, as with the “Complete survey” task type shown in FIG. 6, a drop-down list of types of educational material and/or locations may be presented to the user in field 42 when the task type “Review educational material” is selected in field 40.

As with the task type field 40 above, the value of the parameter to be entered into field 42 may be a numerical or coded value rather than the text indicated in FIG. 5 or 6.

Finally, returning to FIG. 3, as in conventional tasks, there is a field 44 for allowing a description of the task to be specified. As with the task name field 32, this field is free-text, which allows a description of the task or any other required information for the task to be entered. Alternatively, the content of this field 44 could be automatically filled according to the task type and/or task sub-types entered into field 40 and/or 42. For example, where a “Take measurement” task type is specified in field 40 and “weight” is specified in field 42, the task description field 44 could be automatically filled with a description of how to correctly operate the medical device 4 (in this example, e.g., a scale or weight measurement device).

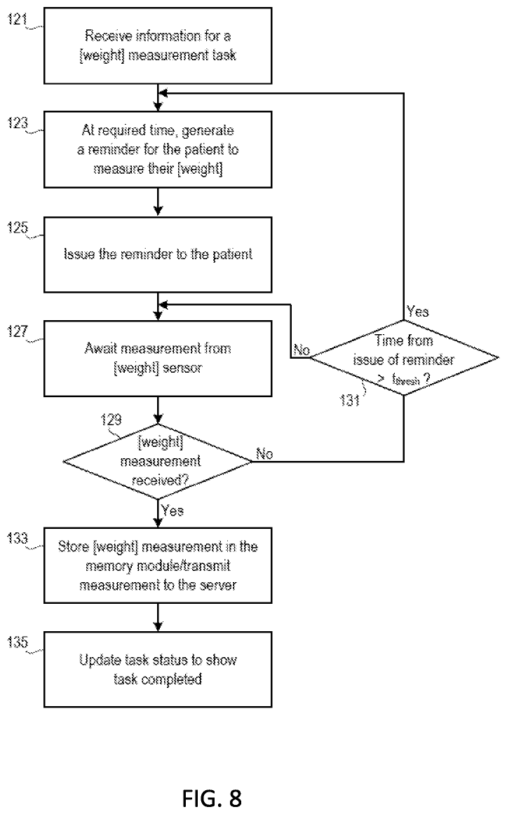

The flow chart in FIG. 8 illustrates a specific embodiment of a method of operating a telehealth hub/patient device 8 for a weight measurement task (i.e. where the task type is “Take measurement” in FIG. 4 and the task sub-type is “weight” in FIG. 5), although it will be appreciated that the method may be readily adapted to other types of measurement (as indicated in FIG. 8 by the parameter “weight” being shown in square brackets).

In a first step, step 121, the patient device 8 receives information for a weight measurement task. That is, the patient device 8 receives information indicating a task type “Take measurement” and a task sub-type “weight”, along with at least a date/time when the measurement is to be taken. The telehealth hub 8 determines the operations associated with this task type and task sub-type (for example by looking up the operations associated with the specified task type and sub-type in the memory module 18), and in particular identifies two specific operations for the patient device 8 to perform to improve the patient experience in completing the task. In particular, when the task is started, the patient device 8 is to automatically monitor a weight sensor of medical device 4 to determine when a measurement has been taken, and when the measurement is made and received by the patient device 8, the patient device 8 is to store the weight measurement and update the task status to indicate that the task has been completed.

Then, in step 123, the patient device 8 generates a reminder for the patient to remind them to measure their weight at the appropriate time. The appropriate time might be the date/time specified in the task information, or some predetermined time before the date/time specified for the completion of the task.

In step 125, the reminder is issued to the patient. Issuing the reminder may comprise displaying a message on the display in the user interface 20 of the patient device 8, flashing lights in the user interface 20, generating an audible sound (e.g. a spoken message or alarm tone), etc.

Then, in step 127, the patient device 8 awaits a weight measurement from the weight sensor of medical device 4. If it is determined in step 129 that no weight measurement has been received, the patient device 8 checks whether a predetermined time tthresh has elapsed since the reminder was issued to the patient (step 131), and if not, the method returns to step 127 and awaits a measurement from the weight sensor of the medical device 4. If the time elapsed since the reminder was issued to the patient exceeds tthresh, the method returns to step 123 and a further reminder for the patient is generated.

When a weight measurement is received from the weight sensor of medical device 4, the method moves to step 133 in which the telehealth hub 8 completes the first operation associated with the specified task type and task sub-type by storing the weight measurement in the memory module 18 and/or transmitting the measurement to the server 22.

Then, the telehealth hub 8 completes the second operation associated with the specified task type and task sub-type and updates the task status to ‘completed’ to indicate that the patient has completed the task (step 135).

Thus, in this embodiment, the definition of a specific task type in the task information and knowledge at the telehealth hub 8 of operations associated with that task type allow the telehealth system 2 to automatically determine when the task has been completed and to automatically complete the task workflow for the patient, thereby reducing the interactions required by the patient with the patient device 8 compared to conventional telehealth systems 2.

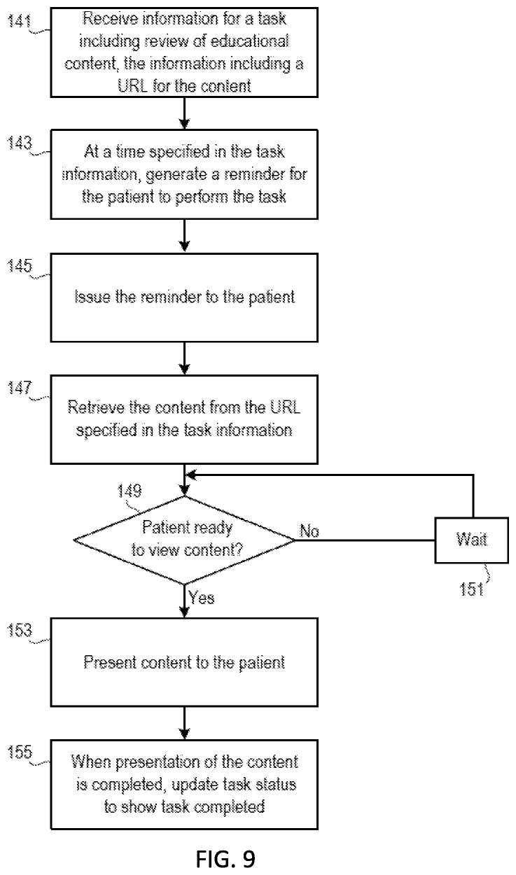

The flow chart in FIG. 9 illustrates a specific embodiment of a method of operating a telehealth hub/patient device 8 for a task involving the review of educational content (i.e. where the task type is “Review educational material” in FIG. 4 and a URL is provided in the task sub-type field 42 in FIG. 7), although it will be appreciated that the method may be readily adapted to other task types, such as completing a survey.

In a first step, step 141, the patient device 8 receives information for a review educational material task. That is, the patient device 8 receives information indicating a task type “Review educational material” and a task sub-type specifying a URL where the educational material is located on the Internet or WAN 14, along with at least a date/time by which the material is to be reviewed. The telehealth hub 8 determines the operations associated with this task type (for example by looking up the operations associated with the specified task type in the memory module 18), and in particular identifies two specific operations for the patient device 8 to perform to improve the patient experience in completing the task. In particular, when the task is started, the patient device 8 is to automatically retrieve the content from the specified URL address, and when the content is played by the patient device 8, the patient device 8 is to automatically update the task status to indicate that the task has been completed.

Thus, in step 143, the patient device 8 generates a reminder for the patient to remind them to review the educational material at the appropriate time. The appropriate time might be the date/time specified in the task information, or some predetermined time before the date/time specified for the completion of the task.

In step 145, the reminder is issued to the patient. Issuing the reminder may comprise displaying a message on the display in the user interface 20 of the patient device 8, flashing lights in the user interface 20, generating an audible sound (e.g. a spoken message or alarm tone), etc.

Then, in step 147, the telehealth hub 8 completes the first operation associated with the specified task type by retrieving the content from the specified URL address and waiting for the patient to indicate that they are ready for the content to be played. If it is determined in step 149 that no positive indication been received from the patient (e.g. the patient has indicated that they are not ready to review the content or no indication has been received, the patient device 8 waits (step 151) and returns to step 149. Alternatively, however, as with the method shown in FIG. 8, the patient device 8 may issue another reminder if a predetermined time period has elapsed.

When the patient indicates that they are ready to view the content, the content is played or presented to the patient (step 153). This step may comprise displaying the content on a display of the user interface 20 and/or playing an audio part of the content through a speaker or speakers in the user interface 20.

When the content has been played to the patient, the method moves to step 155 in which the telehealth hub 8 completes the second operation associated with the specified task type by updating the task status to ‘completed’ to indicate that the patient has completed the task.

Thus, in this embodiment, the definition of a specific task type in the task information and knowledge at the telehealth hub 8 of operations associated with that task type allow the telehealth hub 8 to automatically retrieve the required content to minimize a waiting time for the patient when they are ready to complete the task, and to determine when the task has been completed and update the task workflow for the patient, thereby reducing the interactions required by the patient with the patient device 8 compared to conventional telehealth systems 2.

There is therefore provided an improved way of managing tasks within a patient monitoring or telehealth system 2.

In some embodiments, the generated alert of the medical device/medication dispensing device, and/or the patient's response to the generated alert, may be predicted and/or altered based on aggregated data related to the other users of the system 2, 2A.

An exemplary method 200 for facilitating a response to a generated alert for a patient in accordance with an embodiment is illustrated in FIG. 10. At 202, physiological data (age, gender, and type and dose of medication being dispensed from the dispensing device) related to the patient or user may be obtained by the one or processors. In some embodiments, the system may obtain user profile data of the user, such as data indicating a condition, age, gender, nationality, geographical location, medications (e.g., type and dose of medication being dispensed from the dispensing device), social economics, or other aspect of the user. A plurality of users may be determined as being related to the user based on the user profile data. For example, in method 200, at 204, a plurality of users related to the patient based on the obtained physiological data may be determined by the processor(s). Based on the determination of the plurality of users, aggregated user profile data and/or aggregated response data related to the plurality of users may be obtained. As an example, the aggregated user profile data may indicate conditions of the plurality of users, ages, genders, nationalities, geo graphical locations, medications (types and doses), social economics, or other aspects of the plurality of users, and the aggregated response data may include data indicating actual responses of the users (e.g., to presentation of predicted responses for conditions related to the condition of the user).

In an embodiment, the aggregated response data, also referred to herein as combined response data, includes data indicating actual responses of the plurality of users to presentation of generated alerts including the dispensing triggering element, as shown at 206.

In an embodiment, based on the aggregated user profile data and the aggregated response data, a response for the patient to the generated alert may be generated.

At 208, provide the combined response data related to the plurality of users to a neural network as reference feedback for the neural network's generation of alerts, the neural network configuring one or more layers of the neural network based the combined response data and the predicted generated alerts; subsequent to the configuration of the neural network, provide the physiological data related to the patient to the neural network to predict a response to the generated alert for the patient; and cause, via the medication dispensing device, presentation of the predicted response to the patient.

In some embodiments, as shown at 208, the system/method may include providing the combined response data related to the plurality of users to a neural network or other machine learning models, and the neural network may predict a patient's response to a generated alert. Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it propagates to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

Referring back to FIG. 10, subsequent to the configuration of the neural network, at 210, the physiological data related to the patient may be provided by the system/processor(s) to the neural network to predict a response to the generated alert for the patient. Thereafter, at 212, via the medication dispensing device, the predicted response may be presented to the patient.

In accordance with an embodiment, the method implemented by the processor(s)/system may further include providing response data related to the patient to the neural network as reference feedback for the neural network's generation of alerts. In this case, the response data indicate an actual response of the patient to presentation of the generated alert including the dispensing triggering element. Accordingly, the neural network may further configure one or more layers of the neural network based on the response data and the generated alert.

In this way, for example, the prediction and/or generation of alerts may be personalized for the user without first requiring a significant amount of the patient's response data (e.g., data indicating the patient's actual responses) or without any such user response data as the initial training set for the prediction model.

The actual response data of the plurality of users and/or response(s) from the patient may include, for example, a response to the triggering element of the generated alert wherein a user provides an indication or feedback regarding the date and time for responding to the alert. For example, the response to the triggering element may include that the timing of the generated alert is incorrect, either because the system may detect that the patient/user has not performed an action (e.g., the sensor has detected that the dispensed predetermined dose of medication has not been removed from the medication dispensing device/receiving area of the medication dispensing device; the sensor has detected that the patient has not performed the physical action with the medical device), or the patient/user has indicated that the generated alert is inconvenient, or both. For example, in some cases, the patient may provide a response or feedback to delay the alert and/or time to take the dose of medication, perform the task, etc.

Accordingly, in an embodiment, the response(s) may be used as the reference feedback by the neural network in order to predict and/or update the generated alert (e.g., update the date and time that the alert is generated).

In some embodiments, the system may be configured to personalize the timing for generating the alert and/or taking medication, complete the task, etc. In one embodiment, the scheduled date and time for taking medication, completing the task, etc. may be based pre-set options as a default and/or based on doctor recommendations. In an embodiment, the system may utilize the neural network to develop an algorithm for learning a patient's routine and then generate the alert with the triggering element (and/or reminder(s)) based on a predicted date and time which is a best moment for a patient to execute that task. For example, the system may utilize data motion, heart rate, location, proximity, external light, and/or sleep monitoring patterns as detected by the system, medical device 4, or patient device 8 to determine a patient's routine through a day (e.g., 24 hour period). In an exemplary embodiment, if a patient's physiological data indicates that a dose of medication should be taken with breakfast, then the system may be configured to determine a time that the user gets up from sleeping and is awake, by taking measurements after the patient wakes up. Then, based on such data (which may further include a determined proximity of the patient to the medical/medication device and the medication status), an alert may be generated.

Accordingly, the disclosed system may be configured to tailor scheduling of tasks and generation of alerts to fit patients' routines to eliminate disruption in daily activities.

The system may further predict a best time to generate an alert/reminder(s) for a certain task if that task is to be performed or executed/completed in a certain location via proximity detection (e.g., using Bluetooth and/or GPS).

Further, user input and response data from the patient and/or other users enables adjustment of the generated alerts and predicted responses to said alerts. For example, a predicted response to the generated alert may include requesting input from a patient to ask the user if the time for receiving the alert/reminder and/or completing the task (e.g., taking the dose of medication) is convenient. Based on feedback from the user, adjustments may be made. In some embodiments, an adjustment may be postponing a reminder/alert, such as snoozing a reminder through an interface by a time period (e.g., 10-15 minutes). In another embodiment, the system may be configured to determine that a patient has requested multiple postponements of the reminder/alert. Accordingly, the system may be configured to utilize this response data from the patient to determine that the reminder/alert has not been provided at a most convenient time, and, instead, adjust the scheduled date and time for taking the medication, completing the task, etc.

In another embodiment, contextual information of an environment may be utilized by system to generate alerts/reminders for completing a task, taking medication, etc. For example, if a patient is tasked with an exercise regimen, e.g., go out and have a walk, the system may be configured to determine and learn a time period during the day when it is less crowded and thus direct the patient during the learned time to perform the exercise regimen. In a similar manner, the system may utilize information from an outside source, e.g., a healthcare professional or pharmacist via terminal 24 with regards to a number of people in a pharmacy and/or number of prescription, and determined and learn a time period to alert the patient to go to a selection location (e.g., pharmacy) to complete the task.

In one embodiment, the system may be further configured to compare a completion date and time to the specified date and time frame for completing the taking of the predetermined dose of medication, completing the task, etc.; then determine a difference between the completion date and time and the specified date and time frame; and update the information for taking the predetermined dose of medication, completing the task, etc. to a different date and time frame for completion based on the determined difference.

In accordance with an embodiment, the system 2 may be configured to utilize data from one or more other users that relates to a user or patient to determine and/or predict dates and times to address an assigned task, e.g., to take a predetermined dose of medication, perform a medical action, perform a task, etc.

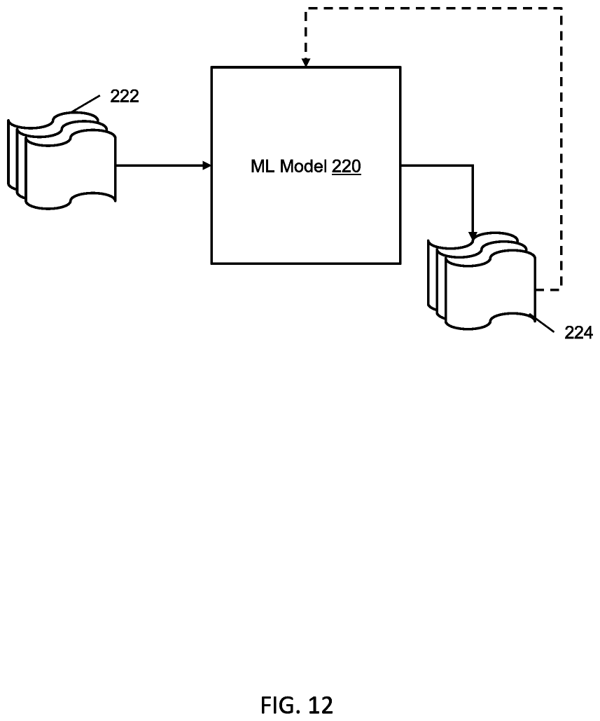

As an example, with respect to FIG. 12, machine learning model 220 may take inputs 222 and provide outputs 224. In some embodiments, outputs 224 may be fed back to machine learning model 220 as input to train machine learning model 220 (e.g., alone or in conjunction with user responses to outputs 224, labels associated with the inputs, or with other reference feedback information). In some embodiments, machine learning model 220 may update its configurations (e.g., weights, biases, or other parameters) based on its assessment of its prediction (e.g., outputs 224) and reference feedback information (e.g., user responses, reference labels, or other information). In some embodiments, where machine learning model 220 is a neural network, connection weights may be adjusted to reconcile differences between the neural network's prediction and the reference feedback. Some embodiments include one or more neurons (or nodes) of the neural network requiring that their respective errors is/are sent backward through the neural network to them to facilitate the update process (e.g., backpropagation of error). Updates to the connection weights may, for example, be reflective of the magnitude of error propagated backward after a forward pass has been completed. In this way, for example, the machine learning model 220 may be trained to generate better predictions.

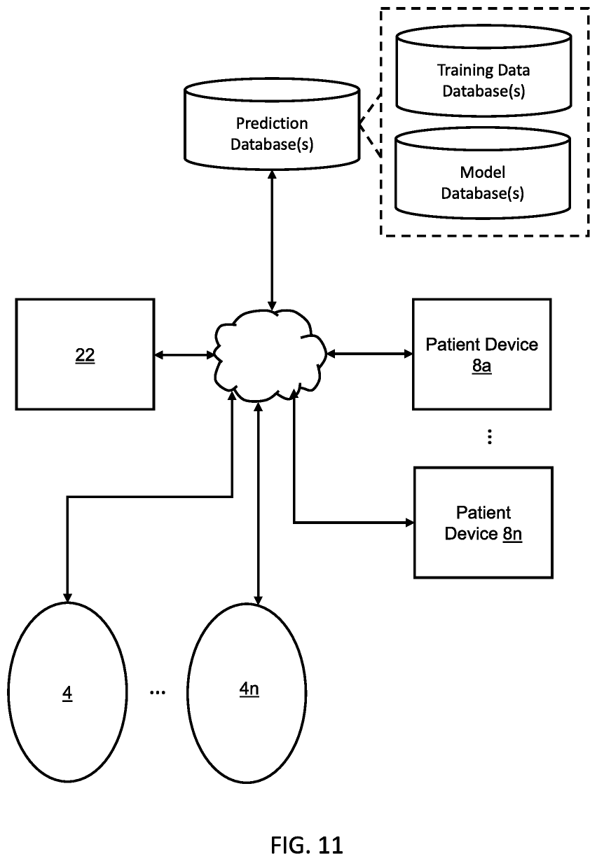

In some embodiments, one or more prediction models may initially be trained or configured to be targeted for one or more sets of user classifications, such as a set of users that fall into one or more of an age group, gender category, nationality, geographical location or region, medication group (e.g., those are under taking certain medications), condition or condition type, social economic class, or other classifications. As an example, such prediction models may be stored in one or more database for later use (e.g., model database(s) 220 of prediction database(s) or other databases, shown in FIG. 11). Based on a determination that a user falls under a particular set of user classifications, a prediction model initially trained or configured to be targeted for that set of user classifications may be selected, and an instance of the prediction model may obtained and further trained or configured based on the user profile data of the user to facilitate a response prediction for the user.

To further personalize the experience and accuracy for the user, an instance of the prediction model may be obtained and furthered trained or configured using data pertaining to the user (e.g., user profile and response data of the user) and aggregated data pertaining to users that are similar to the user (e.g., aggregated user profile data and combined response data of the similar users). In this way, for example, such prediction models may be specifically personalized for a given user, while reducing the amount of time and computational resources necessary to perform such personalization. As an example, because a prediction model is already pre-trained or configured for a larger set of users that already fall under one or more of the same classifications of the user, further personalization of the prediction model may be performed using a smaller training data set (e.g., smaller input training data, smaller reference feedback training data, etc. from a training data database) than would otherwise have been required to achieve similar accuracy of predictions.

In accordance with embodiments herein, the mechanism of these tasks may be similar to how office productivity tools such as Microsoft Outlook™ manage tasks. These tools enable users or the system to create and use tasks to structure their working activities. Common parameters of tasks include an owner (i.e. who the task is for), the start date, the due date (i.e. when the task must be completed by), priority (i.e. how important the task is), status (i.e. not yet completed, completed, etc.), title, summary and description. These tools often semantically understand the parameters such as owner (which may be defined in terms of a system username or email address, etc.), start/due date, priority and status since they may have a predetermined format and/or a predetermined list of options (e.g. low priority, medium priority, high priority, etc.). Due to this semantic understanding, the tools may take actions in respect of these parameters (for example issuing a reminder to the owner as the due date approaches). However, the title and description fields, which typically indicate the details of the task the patient is to perform, and in some cases how the task is to be performed, are ‘free text’ fields, meaning that there are little or no restrictions on their format or content, and the tools are not able to understand or use the information in these fields.

Thus, these tools are able to help a patient with the timing of a task, but is not able to support the patient with the completion of the task itself. The tools are also not able to verify that the patient-selected status of a task (e.g. completed) is correct, or vice versa. Therefore, there is a need for an improved way of managing tasks within a patient monitoring or telehealth system.

According to one aspect of the disclosure, there is provided a method of operating a patient device in a patient monitoring system to assist a patient in completing a task, there being a predefined set of types of task for the patient to complete, the method comprising receiving information on a task for the patient to complete, the information comprising an indication of the type of task from the predefined set the patient is to complete; determining one or more operations associated with the type of task indicated in the received information that the patient device is to perform on initiation of the task or on occurrence of a predefined event; and on initiation of the task by the patient or on occurrence of the predefined event, performing the one or more operations associated with the task.

In some embodiments, the step of receiving information on the task for the patient to complete comprises receiving the information from a server in the patient monitoring system.

In other embodiments, the step of receiving information on the task for the patient to complete comprises retrieving the information from a memory in the patient device.

In some embodiments, the patient device has information on the operation or operations associated with each type of task in the predefined set stored in a memory, and wherein the step of determining the one or more operations comprises looking up the operation or operations associated with the type of task indicated in the received information.

In other embodiments, the information received on the task includes information indicating the one or more operations the patient device is to perform, and wherein the step of determining the one or more operations comprises reading the indication of the one or more operations contained in the received information.

In particular embodiments, the operation or operations the patient device is to perform comprises any one or more of starting a workflow to guide the patient through the task that the patient is to complete, moving a workflow on to a subsequent step or closing the workflow on detection of the completion of a predetermined action by the patient, placing an input cursor on a display of the patient device in an appropriate field, issuing instructions to the patient regarding the performance or completion of the task, issuing one or more reminders to the patient regarding the task and retrieving a survey, questionnaire, educational material or other content from a memory of the patient device or other storage location.

In some embodiments, the received information on the task further comprises information indicating a specific action or task sub-type related to the indicated type of task.

In preferred embodiments, the predefined set of types of task for the patient to complete comprises one or more of taking a measurement of a physiological characteristic, completing a survey or questionnaire, reviewing educational content and taking a dose of medication.

In particular embodiments, when the indicated type of task indicates the patient is to take a measurement of a physiological characteristic, the received information on the task further comprises additional information indicating a specific action or task sub-type related to the measurement of a physiological characteristic, the additional information indicating the particular physiological characteristic of the patient that is to be measured.

In particular embodiments, when the indicated type of task indicates the patient is to complete a survey or questionnaire, the received information on the task further comprises additional information indicating a specific action or task sub-type related to the survey or questionnaire, the additional information indicating the type of survey or questionnaire to be completed and/or a location or address at which the survey or questionnaire is stored.

In particular embodiments, when the indicated type of task indicates the patient is to review educational content, the received information on the task further comprises additional information indicating a specific action or task sub-type related to the educational content, the additional information indicating the type of content to be reviewed and/or a location or address at which the content is stored.

In particular embodiments, when the indicated type of task indicates the patient is to take a dose of medication, the received information on the task further comprises additional information indicating a specific action or task sub-type related to the dose of medication, the additional information indicating the type of medication to be taken and/or the amount of medication in the dose.

The indication of the type of task the patient is to complete may comprise a numerical, text-based or coded value in a ‘task type’ field in a data structure used to define a task, with each type of task in the predefined set having a respective value associated therewith.

In some embodiments, the system is configured to detect routines of user and then tailor the scheduled date and time for completion of task to a user's routine.

According to another aspect of the disclosure, there is provided a computer program product having computer-readable code embodied therein, the computer-readable code being configured such that, on execution by a suitable computer or other electronic device, the computer or electronic device performs a method as described above.

According to yet another aspect of the disclosure, there is provided a control unit for a patient device in a patient monitoring system, wherein the control unit is configured to receive information on a task for the patient to complete, the information comprising an indication of the type of task the patient is to complete, there being a predefined set of types of task; determine one or more operations associated with the type of task indicated in the received information that the patient device is to perform on initiation of the task or on occurrence of a predefined event; and perform the one or more operations associated with the task on initiation of the task by the patient or on determining that the predefined event has occurred.

In some embodiments, the control unit is configured to receive the information on the task for the patient to complete from a server in the patient monitoring system.

In other embodiments, the patient device has information on the task for the patient to complete stored in a memory, and the control unit is configured to retrieve the information on the task for the patient to complete from the memory.

In some embodiments, the patient device has information on the operation or operations associated with each type of task in the predefined set stored in a memory, and the control unit is configured to determine the one or more operations by looking up the operation or operations associated with the type of task indicated in the received information.

In other embodiments, the information received on the task includes information indicating the one or more operations the patient device is to perform, and the control unit is configured to determine the one or more operations by reading the indication of the one or more operations contained in the received information.

In particular embodiments, the operation or operations the patient device is to perform comprises any one or more of starting a workflow to guide the patient through the task that the patient is to complete, moving a workflow on to a subsequent step or closing the workflow on detection of the completion of a predetermined action by the patient, placing an input cursor on a display of the patient device in an appropriate field, issuing instructions to the patient regarding the performance or completion of the task, issuing one or more reminders to the patient regarding the task and retrieving a survey, questionnaire, educational material or other content from a memory of the patient device or other storage location.

In some embodiments, the received information on the task further comprises information indicating a specific action or task sub-type related to the indicated type of task.

In preferred embodiments, the predefined set of types of task for the patient to complete comprises one or more of taking a measurement of a physiological characteristic, completing a survey or questionnaire, reviewing educational content and taking a dose of medication.

In particular embodiments, when the indicated type of task indicates the patient is to take a measurement of a physiological characteristic, the received information on the task further comprises additional information indicating a specific action or task sub-type related to the measurement of a physiological characteristic, the additional information indicating the particular physiological characteristic of the patient that is to be measured.

In particular embodiments, when the indicated type of task indicates the patient is to complete a survey or questionnaire, the received information on the task further comprises additional information indicating a specific action or task sub-type related to the survey or questionnaire, the additional information indicating the type of survey or questionnaire to be completed and/or a location or address at which the survey or questionnaire is stored.

In particular embodiments, when the indicated type of task indicates the patient is to review educational content, the received information on the task further comprises additional information indicating a specific action or task sub-type related to the educational content, the additional information indicating the type of content to be reviewed and/or a location or address at which the content is stored.

In particular embodiments, when the indicated type of task indicates the patient is to take a dose of medication, the received information on the task further comprises additional information indicating a specific action or task sub-type related to the dose of medication, the additional information indicating the type of medication to be taken and/or the amount of medication in the dose.

The indication of the type of task the patient is to complete may comprise a numerical, text-based or coded value in a ‘task type’ field in a data structure used to define a task, with each type of task in the predefined set having a respective value associated therewith.

According to still yet another aspect of the disclosure, there is provided a patient monitoring system, comprising a patient device comprising a control unit as described above; and a server that is configured to generate information on a task for the patient to complete, the information indicating a type of task from the predefined set of task types, and to communicate the information on the task to the patient device.

Furthermore, the different embodiments disclosed herein may take the form of a computer program product accessible from a computer usable or computer readable medium providing program code for use by or in connection with a computer or any device or system that executes instructions. For the purposes of this disclosure, a computer usable or computer readable medium may generally be any tangible device or apparatus that may contain, store, communicate, propagate, or transport the program for use by or in connection with the instruction execution device.

In so far as embodiments of the disclosure have been described as being implemented, at least in part, by software-controlled data processing devices, it will be appreciated that the non-transitory machine-readable medium carrying such software, such as an optical disk, a magnetic disk, semiconductor memory or the like, is also considered to represent an embodiment of the present disclosure.

The computer usable or computer readable medium may be, for example, without limitation, an electronic, magnetic, optical, electromagnetic, infrared, or semiconductor system, or a propagation medium. Non-limiting examples of a computer readable medium include a semiconductor or solid-state memory, magnetic tape, a removable computer diskette, a random access memory (RAM), a read-only memory (ROM), a rigid magnetic disk, and an optical disk. Optical disks may include compact disk-read only memory (CD-ROM), compact disk-read/write (CD-R/W), and DVD.

Further, a computer usable or computer readable medium may contain or store a computer readable or usable program code such that when the computer readable or usable program code is executed on a computer, the execution of this computer readable or usable program code causes the computer to transmit another computer readable or usable program code over a communications link. This communications link may use a medium that is, for example, without limitation, physical or wireless.

A data processing system or device suitable for storing and/or executing computer readable or computer usable program code will include one or more processors coupled directly or indirectly to memory elements through a communications fabric, such as a system bus. The memory elements may include local memory employed during actual execution of the program code, bulk storage, and cache memories, which provide temporary storage of at least some computer readable or computer usable program code to reduce the number of times code may be retrieved from bulk storage during execution of the code.

Input/output, or I/O devices, may be coupled to the system either directly or through intervening I/O controllers. These devices may include, for example, without limitation, the interface, keyboards, touch screen displays, and pointing devices. Different communications adapters may also be coupled to the system to enable the data processing system to become coupled to other data processing systems, remote printers, or storage devices through intervening private or public networks. Non-limiting examples are modems and network adapters and are just a few of the currently available types of communications adapters.

Although example embodiments have been described in detail for the purpose of illustration, it is to be understood that such detail is solely for that purpose and that embodiments are not limited to the disclosed embodiments, but, on the contrary, are intended to cover modifications and equivalent arrangements that are within the scope of the appended claims. For example, it is to be understood that embodiments contemplate that, to the extent possible, one or more features of any embodiment can be combined with one or more features of any other embodiment.

As used throughout this application, the word “may” is used in a permissive sense (i.e., meaning having the potential to), rather than the mandatory sense (i.e., meaning must). The words “comprise,” “comprising,” “comprises,” “include”, “including”, and “includes” and the like mean including, but not limited to. As used throughout this application, the singular forms “a,” “an,” and “the” include plural referents unless the context clearly indicates otherwise, and notwithstanding the use of other terms and phrases for one or more elements, such as “one or more.” The term “or” is non-exclusive (i.e., encompassing both “and” and “or”), unless the context clearly indicates otherwise. Further, unless otherwise indicated, statements that one value or action is “based on” another condition or value encompass both instances in which the condition or value is the sole factor and instances in which the condition or value is one factor among a plurality of factors. Unless the context clearly indicates otherwise, statements that “each” instance of some collection have some property should not be read to exclude cases where some otherwise identical or similar members of a larger collection do not have the property, i.e., each does not necessarily mean each and every.

While the disclosure has been illustrated and described in detail in the drawings and foregoing description, such illustration and description are to be considered illustrative or exemplary and not restrictive; the disclosure is not limited to the disclosed embodiments.

Variations to the disclosed embodiments can be understood and effected by those skilled in the art in practicing the claimed disclosure, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

While the principles of the disclosure have been made clear in the illustrative embodiments set forth above, it will be apparent to those skilled in the art that various modifications may be made to the structure, arrangement, proportion, elements, materials, and components used in the practice of the disclosure.

It will thus be seen that the features of this disclosure have been fully and effectively accomplished. It will be realized, however, that the foregoing preferred specific embodiments have been shown and described for the purpose of illustrating the functional and structural principles of this disclosure and are subject to change without departure from such principles. Therefore, this disclosure includes all modifications encompassed within the spirit and scope of the following claims.

What is claimed is:

1. A medication dispensing system for facilitating dispensing of medication, the system comprising:
one or more processors executing computer program instructions that, when executed, cause the one or more processors to:
determine a proximity of a patient to a medication dispensing device associated with the patient, the medication dispensing device being configured to dispense a medication for the patient;
determine a medication status associated with the patient, the medication status being related to the medication that is dispensed by the medication dispensing device;
generate, via the medication dispensing device, an alert including a dispensing triggering element based on the proximity of the patient and the medication status such that:
(i) the alert includes a first alarm type in response to the proximity satisfying a proximity threshold and the medication status satisfying a first timing threshold;
(ii) the alert includes a second alarm type in response to the proximity satisfying the proximity threshold and the medication status satisfying a second timing threshold different from the first timing threshold;
detect patient activation of the dispensing triggering element of the alert;
instruct, based on the detection, the medication dispensing device to dispense a predetermined dose of medication from a storage area of the medication dispensing device;
monitor, via one or more sensors of the medication dispensing device, the medication dispensing device for occurrence of the dispensing of the predetermined dose and occurrence of a removal of the dispensed predetermined dose of the medication dispensing device; and
update, based on the monitoring indicating occurrence of the removal of the predetermined dose, the medication status associated with the patient.

2. The medication dispensing system according to claim 1, wherein the medication dispensing device includes a receiving area, and wherein, based on the detection, the one or more processors instruct dispensing from the storage area to the receiving area of the medication dispensing device.

3. The medication dispensing system according to claim 2, wherein the one or more sensors are configured to monitor the medication dispensing device to determine removal of the predetermined dose from the receiving area.

4. The medication dispensing system according to claim 1, wherein the one or more processors are further configured to:
establish communication with a patient device associated with the patient, and
determine the proximity of the patient device to the medication dispensing device via the sensor of the medication dispensing device.

5. The medication dispensing system according to claim 4, wherein one of the first alarm type and the second alarm type is issued via the patient device.

6. The medication dispensing system according to claim 5, wherein the other of the first alarm type and the second alarm type is issued using the medication dispensing device.

7. The medication dispensing system according to claim 1, wherein one of the first alarm type and the second alarm type comprises an audio alert, and wherein the other comprises a visual alert.

8. The medication dispensing system according to claim 1, wherein the one or more processors are further configured to:
obtain physiological data related to the patient, the physiological data including age, gender, and type and dose of medication being dispensed by the medication dispensing device;
determine a plurality of users related to the patient based on the obtained physiological data;
obtain, based on the plurality of users, combined response data indicating actual responses of the plurality of users to presentation of generated alerts including the dispensing triggering element;
provide the combined response data related to the plurality of users to a neural network as reference feedback for the neural network's generation of alerts, the neural network configuring one or more layers of the neural network based the combined response data and the predicted generated alerts;
subsequent to the configuration of the neural network, provide the physiological data related to the patient to the neural network to predict a response to the generated alert for the patient; and
cause, via the medication dispensing device, presentation of the predicted response to the patient.

9. The medication dispensing system according to claim 8, wherein the one or more processors are caused to:
provide response data related to the patient to the neural network as reference feedback for the neural network's generation of alerts, the response data indicating an actual response of the patient to presentation of the generated alert including the dispensing triggering element, the neural network configuring one or more layers of the neural network based on the response data and the generated alert.

10. A method comprising:
determining a proximity of a patient to a medical device, the medical device being arranged such that the patient performs a physical action therewith;
determining a medical status associated with the patient, the medical status being related to the physical action for the patient to initiate with the medical device;
generating an alert including a triggering element based on the proximity of the patient and the medical status such that:
(i) the alert includes a first alarm type in response to the proximity satisfying a proximity threshold and the medical status satisfying a first timing threshold;
(ii) the alert includes a second alarm type in response to the proximity satisfying the proximity threshold and the medical status satisfying a second timing threshold different from the first timing threshold;
detecting patient activation of the triggering element of the alert;
monitoring, via one or more sensors of the medical device, initiation and occurrence of the physical action by the patient using the medical device; and
updating, based on the monitoring indicating occurrence of the physical action, the medical status associated with the patient.

11. The method according to claim 10, wherein one of the first alarm type and the second alarm type comprises an audio alert, and wherein the other comprises a visual alert.

12. The method according to claim 10, further comprising:
establishing communication with a patient device associated with the patient, and
determining the proximity of the patient device to the medical device via the sensor of the medical device.

13. The method according to claim 12, wherein one of the first alarm type and the second alarm type is issued via the patient device.

14. The method according to claim 13, wherein the other of the first alarm type and the second alarm type is issued using the medical device.

15. The method according to claim 10, wherein one of the first alarm type and the second alarm type comprises an audio alert, and wherein the other comprises a visual alert.

16. One or more non-transitory computer-readable media comprising one or more instructions that, when executed by one or more processors, cause operations comprising:
determining a proximity of a patient to a medical device, the medical device being arranged such that the patient performs a physical action therewith;
determining a medical status associated with the patient, the medical status being related to the physical action for the patient to initiate with the medical device;
generating an alert including a triggering element based on the proximity of the patient and the medical status;
detecting patient activation of the triggering element of the alert;
monitoring, via one or more sensors of the medical device, initiation and occurence of the phsyical action by the patient using the medical device; and
updating, based on the monitoring indicating occurrence of the phsyical action, the medical status associated with the patient.

17. The one or more non-transitory computer-readable media according to claim 16, wherein the generation of the alert comprises:
(i) the alert includes a first alarm type in response to the proximity satisfying a proximity threshold and the medical status satisfying a first timing threshold;
(ii) the alert includes a second alarm type in response to the proximity satisfying the proximity threshold and the medical status satisfying a second timing threshold different from the first timing threshold.

18. The one or more non-transitory computer-readable media according to claim 17, further configured to, when executed by one or more processors, cause operations comprising:
establishing communication with a patient device associated with the patient, and
determining the proximity of the patient device to the medical device via the sensor of the medical device.

19. The one or more non-transitory computer-readable media according to claim 18, wherein one of the first alarm type and the second alarm type is issued via the patient device.

20. The one or more non-transitory computer-readable media according to claim 19, wherein the other of the first alarm type and the second alarm type is issued using the medical device.
